(19) United States
(12) Patent Application Publication
(10) Pub. No.: US 2013/0109730 A1
(43) Pub. Date: May 2, 2013

(54) TREATMENT OF LIVER DISEASES IN WHICH IRON PLAYS A ROLE IN PATHOGENESIS

(71) Applicant: Novartis AG, Basel (CH)

(72) Inventors: Daniele Alberti, Monza (IT); Peter Marks, Randolph, NJ (US); Hanspeter Nick, Duggingen (CH); Lisa Grace Rojkjaer, Princeton, NJ (US)

(73) Assignee: Novartis AG, Basel (CH)

(21) Appl. No.: 13/721,196

(22) Filed: Dec. 20, 2012

Related U.S. Application Data
(63) Continuation of application No. 12/641,690, filed on Dec. 18, 2009, now abandoned.
Application No. 12/641,690 is a continuation of application No. 11/913,678, filed on Nov. 6, 2007, now abandoned.
(60) Provisional application No. 60/685,848, filed on May 31, 2005.
(60) Provisional application No. 60/692,808, filed on Jun. 22, 2005.
(60) Provisional application No. 60/746,786, filed on May 9, 2006.

(51) Int. Cl. A61K 31/4196

(52) U.S. Cl. CPC A61K 31/4196
USPC 514/383

(57) ABSTRACT

The invention relates to the use of 4-[3,5-Bis-(2-hydroxyphenyl)-[1,2,4]-triazol-1-yl]benzoic acid (hereinafter referred to as “Compound I”) for the manufacture of pharmaceutical compositions for the treatment of liver diseases in humans in which iron plays a role in pathogenesis, including viral diseases, such as chronic hepatitis C, optionally in conjunction with antiviral agents and for the treatment of non viral diseases, such as non-alcoholic steatohepatitis and non-alcoholic fatty liver disease.

The invention relates to the use of an iron chelator such as deferiprone (L1), deferitrin, and 4-[3,5-Bis-(2-hydroxyphenyl)-[1,2,4]-triazol-1-yl]benzoic acid (hereinafter referred to as “Compound I”) or pharmaceutically acceptable salts thereof, for the manufacture of pharmaceutical compositions for the prevention and/or treatment of liver diseases, e.g. in humans, in which iron plays a role in pathogenesis, including viral diseases, such as hepatitis B, C, D, G, E, chronic hepatitis C virus infection, cytomegalo virus infection, HIV infection and non viral diseases, such as non-alcoholic steatohepatitis and non-alcoholic fatty liver disease, and liver cancer, such as liver adenocarcinoma, e.g. hepatocellular carcinoma, also called hepatocarcinoma, and to the prevention of progression of said diseases.

### BACKGROUND OF THE INVENTION

Liver disease is among the top ten causes of death in the United States, responsible for over 30,000 deaths annually, see e.g. Vong S, et al. Hepatology; 2004, 39:476-483.

Chronic infection with hepatitis C is a leading cause of liver disease and is a major cause of liver fibrosis and cirrhosis. It is also associated with the development of hepatocellular carcinoma in a percentage of infected individuals. The current standard of care, treatment with interferon and ribavirin, produces virologic remissions in only about half of patients treated.

Non-alcoholic steatohepatitis is a metabolic syndrome associated with fibrosis of the liver and progression to cirrhosis in about 20% of cases, see e.g. Ong et al. Am. J Gastroenterol 2003, 98:1915-1917. The current standard of care, control of metabolic parameters and weight loss, is effective in a minority of patients. Nonalcoholic steatohepatitis or NASH is a common, often “silent” liver disease. It resembles alcoholic liver disease, but occurs in people who drink little or no alcohol. The major feature in NASH is fat in the liver, along with inflammation and damage. Most people with NASH feel well and are not aware that they have a liver problem. Nevertheless, NASH can be severe and can lead to cirrhosis, in which the liver is permanently damaged and scarred and no longer able to work properly. NASH affects 2 to 5 percent of Americans.

Non-alcoholic fatty liver disease (NAFLD) is a common cause of elevated liver function tests and is marked histologically by deposition of fat, primarily macrovesicular, in hepatocytes. Although a benign disorder in the majority of instances, up to 20% of patients with NAFLD have nonalcoholic steatohepatitis (NASH), and can progress to cirrhosis, liver failure and hepatocellular carcinoma. Several risk factors for NAFLD have been identified, including elevated body mass index (BMI), type 2 diabetes mellitus, advancing age and hypertriglyceridemia. The pathophysiologic basis of NAFLD is thought to be insulin resistance. Most experts consider NAFLD to be the hepatic manifestation of the metabolic syndrome, which includes persons with some combination of insulin resistance, obesity, hypertension and dyslipidemia. Patients with NAFLD develop resistance to insulin.

Compound I is 4-[3,5-Bis-(2-hydroxyphenyl)-[1,2,4]-triazol-1-yl]benzoic acid having the following formula

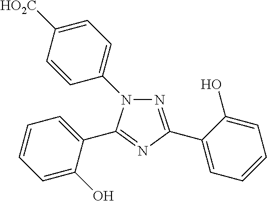 [SMILES: O=C(O)c1ccc(-n2nc(-c3ccccc3O)nc2-c2ccccc2O)cc1]

Compound I in the free acid form, salts thereof and its crystalline forms are disclosed in U.S. Pat. No. 6,465,504 B1. Compound I corresponds to the active moiety.

Compound I is an iron chelator that has been shown to be effective in the selective removal of iron in model systems and in humans, see e.g. Hershko C, et al. Blood. 2001, 97:1115-1122; Nisbet Brown E et al. Lancet. 2003, 361:1597-1602.

However, Compound I was not known to be efficient in the treatment of liver diseases mentioned above. Particularly, there was a need to find an alternative treatment for liver diseases, e.g. liver diseases in which iron plays a role, for example liver diseases due to viral infections, e.g. chronic hepatitis C. In addition, there was a need to find a treatment for liver diseases, e.g. chronic hepatitis C, that are refractory to, non-responsive to or not adequately treated by or non-sustained controlled by, standard therapies, e.g. interferon and ribavirin treatment.

Hereinafter by “Compound I” unless otherwise specified, is meant Compound I free acid form, pharmaceutically acceptable salts thereof, and its crystalline forms.

Deferitrin of the following formula (4S)-2-(2,4-dihydroxyphenyl)-4-methyl-4,5-dihydro-1,3-thiazole-4-carboxylic acid

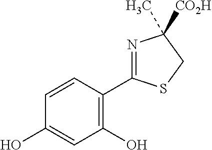 [SMILES: C[C@]1(C(=O)O)CSC(c2ccc(O)cc2O)=N1]

and its process of manufacture is disclosed in WO00/12493, published Mar. 9, 2000.

Deferiprone of the following formula 3-hydroxy-1,2-dimethyl-4-(1,4)pyridinone and its pharmaceutically acceptable preparations are disclosed in EP093498 B1.

The inventors have demonstrated that Compound I can be used to remove iron from the body and propose that removal of iron, e.g. removal of iron to states of near-deficiency or deficiency will be beneficial in certain liver diseases, e.g. viral liver disease, such as hepatitis B, C, D, G, E, chronic hepatitis C virus infection, cytomegalo virus infection, HIV infection, non-alcoholic steatohepatitis and non-alcoholic fatty liver disease, the benefit demonstrated, but not limited to, prevention or reduction in hepatic fibrosis and/or cirrhosis.

The inventors have demonstrated that Compound I can be used to remove iron from the body, e.g. from the liver, and propose that removal of iron, e.g. removal of iron to states of near-deficiency or deficiency, in conjunction with the subsequent or concomitant administration of anti-viral agents, such as, but not limited to, a biologic response modifier, e.g. cytokine, e.g. interferon, e.g. alpha-interferon and/or a nucleoside anti-metabolite, e.g. ribavirin, will be beneficial in certain liver diseases, e.g. viral liver disease, such as hepatitis B, C, D, G, E, chronic hepatitis C virus infection, cytomegalo virus infection, HIV infection, non-alcoholic steatohepatitis and non-alcoholic fatty liver disease, the benefit demonstrated, but not limited to, prevention or reduction in hepatic fibrosis and/or cirrhosis.

Iron state of near-deficiency or deficiency is meant as the liver iron content being below the normal value, especially below 0.5 mg of iron per g of liver dry weight. By normal value is meant an iron content of 0.5 to 1.5 mg of iron per g of liver dry weight. For example a liver iron content of 0.4 mg/g liver dry weight corresponds to a iron state of near-deficiency or deficiency according to the present invention. Iron state of near-deficiency or deficiency can also be monitored by measuring the ferritin level. Blood ferritin concentrations of about 10 to 30 ng per ml of blood correspond the normal ferritin levels. For example a blood ferritin concentration of 5 ng per ml of blood is considered as corresponding to an iron state of near-deficiency or deficiency.

Biological response modifiers, also referred to as cytokines, comprise a group of products that alter immune defenses to enhance, direct or restore the body's ability to fight disease. Biological response modifiers included are for example:

Colony stimulating factors (granulocyte-colony stimulating factors)—G-CSFs,

Granulocyte macrophage-colony stimulating factors—GM-CSFs,

Stem cell growth factors (SCGF),

Erythropoietins, interferons, interleukins (ILs),

Tumor necrosis factor (TNF) inhibitors, and

Peptide thymosin alpha 1, also called thymalfasin, ZADAXIN®.

According to the present invention the biologic response modifier is preferably interferon.

By “nucleoside anti-metabolite” is meant a nucleoside anti-metabolite drug that interfere with duplication of viral genetic material. The “nucleoside anti-metabolites” according to the present invention are not limited to, e.g. ribavirin of the following formula 1-(β-D-Ribofuranosyl)-1H-1,2,4-triazole-3-carboxamide or viramidine, i.e. ICN3142 of the following formula 1-[(2R,3R,4S,5S)-3,4-dihydroxy-5-(hydroxymethyl)oxolan-2-yl]-1,2,4-triazole-3-carboximidamide (also commonly 1-(β-D-Ribofuranosyl)-1,2,4-triazole-3-carboximide) from Valeant Pharmaceuticals International, or valopicitabine, i.e. NM283 (Indenix Pharmaceutical, Inc.).

### BRIEF SUMMARY OF THE INVENTION

The invention relates to the use of Compound I or deferitrin or deferiprone for the treatment of liver diseases in which iron plays a role in pathogenesis, e.g. for the treatment of liver diseases in which iron plays a role in pathogenesis leading to fibrosis and/or cirrhosis and/or the development of liver cancer, such as liver adenocarcinoma, e.g. hepatocellular carcinoma.

The present invention further pertains to the use of Compound I or deferitrin or deferiprone for the manufacture of a medicament for the treatment of liver diseases in which iron plays a role in pathogenesis, leading to fibrosis and/or cirrhosis and/or hepatitis, e.g. viral liver disease, such as hepatitis B, C, D, G, E, chronic hepatitis C virus infection, e.g. chronic hepatitis virus of genotype 1, 2, 3, 4 or 5, cytomegalo virus infection, HIV infection.

The present invention further pertains to the use of Compound I or deferitrin or deferiprone for the manufacture of a medicament for the treatment of liver diseases in which iron plays a role in pathogenesis, leading to fibrosis and/or cirrhosis and/or hepatitis, e.g. non viral liver diseases, such as non-alcoholic steatohepatitis and non-alcoholic fatty liver disease.

The present invention further pertains to the use of Compound I or deferitrin or deferiprone for the manufacture of a medicament for the treatment of liver diseases in which iron plays a role in pathogenesis, leading to fibrosis and/or cirrhosis and/or hepatitis, e.g. viral liver disease, such as hepatitis B, C, D, G, E, chronic hepatitis C virus infection, cytomegalo virus infection, HIV infection in conjunction with the subsequent or concomitant administration of anti-viral agents, e.g. such as a biologic response modifier such as an interferon, e.g. IFNα, pegylated interferon, and/or a nucleoside anti-metabolite, e.g. ribavirin.

The pharmaceutical compositions according to the present invention can be prepared in a manner known per se and are those suitable for enteral, such as oral, and parenteral administration to warm-blooded animals, including man, comprising a therapeutically effective amount of at least one pharmacologically active ingredient, alone or in combination with one or more pharmaceutically acceptable carriers, especially suitable for enteral or parenteral application. The preferred route of administration of the dosage forms of the present invention is orally. Oral formulations of Compound I are disclosed in the following International Patent Application publication WO97/49395 and WO 2004/035026.

The invention relates to a method of treating a warm-blooded animal, e.g. human, with liver disease in which iron plays a role in pathogenesis comprising administering to said animal in need for such a treatment Compound I or deferitrin or deferiprone, in a quantity which is therapeutically effective to remove iron followed by or concomitant with the administration of antiviral agents in the case of hepatitis C, e.g. chronic hepatitis C, or with or without concomitant other therapies in the case of non-alcoholic steatohepatitis.

The invention relates to a method for administering to a human subject suffering from liver disease in which iron plays a role in pathogenesis, Compound I or deferitrin or deferiprone.

In one embodiment of the invention, Compound I is formulated as a dispersible tablet.

In one embodiment of the invention, Compound I is in the polymorphic form A.

In one embodiment of the invention, Compound I is in the polymorphic form A and is formulated as a dispersible tablet.

The invention relates to the use of Compound I or deferitrin or deferiprone for the preparation of a medicament for the treatment of a liver disease, such as a viral liver disease, e.g. chronic hepatitis C, which is refractory to or non-responsive to or not adequately controlled by, non-sustained responsive to, a biologic response modifier treatment, e.g. IFN treatment, e.g. IFN alpha treatment or the combination of a biologic response modifier, e.g. IFN and a nucleoside anti-metabolite, e.g. ribavirin.

The present invention relates to a commercial package comprising Compound I together with instructions for administering said compound to patients having a liver disease, e.g. a viral liver disease, e.g. chronic hepatitis C.

The present invention also pertains to a combination such as a combined preparation or a pharmaceutical composition which comprises (a) an iron chelator, and (b) a biologic response modifier and/or a nucleoside anti-metabolite.

The present invention also pertains to a combination such as a combined preparation or a pharmaceutical composition which comprises (a) an iron chelator selected from the group consisting of Compound I, deferitrin and deferiprone and (b) a biologic response modifier and/or a nucleoside anti-metabolite.

The present invention further pertains to a combination such as a combined preparation or a pharmaceutical composition which comprises (a) an iron chelator being Compound I or deferitrin and (b) a biologic response modifier and/or a nucleoside anti-metabolite.

In one embodiment, the present invention relates to a combination which comprises (a) Compound I and (b) a biologic response modifier and/or a nucleoside anti-metabolite.

In a further embodiment, the present invention relates to a combination which comprises (a) Compound I and (b) an interferon selected from the group comprising Interferon alfa-2a interferon alpha-2b, interferon alfacon-1, peginterferon alpha-2b or peginterferon alpha-2a and/or a nucleoside anti-metabolite selected from the group comprising ribavirin, viramidine or valopicitabine.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single.

The present invention further relates to the use of said combination for the preparation of a medicament for the treatment of liver diseases in which iron plays a role in pathogenesis, leading to fibrosis and/or cirrhosis and/or hepatitis, e.g. viral liver disease, such as hepatitis B, C, D, G, E, chronic hepatitis C virus infection, cytomegalo virus infection, HIV infection, preferably chronic hepatitis C.

The present invention relates to a commercial package comprising Compound I together with an antiviral agent selected from the group consisting of a biologic response modifier, e.g. interferon, e.g. interferon alpha and a nucleoside anti-metabolite, e.g. ribarivin.

The present invention relates to a commercial package comprising Compound I together with instructions to administer Compound I together with at least one antiviral agent selected from the group consisting of a biologic response modifier, e.g. interferon, e.g. interferon alpha and a nucleoside anti-metabolite, e.g. ribarivin.

The present invention relates to the use of deferitrin for the treatment of liver diseases in which iron plays a role in pathogenesis, e.g. for the treatment of liver diseases in which iron plays a role in pathogenesis leading to fibrosis and/or cirrhosis and/or the development of liver cancer, such as liver adenocarcinoma, e.g. hepatocellular carcinoma.

The present invention relates to the use of deferitrin for the treatment of a viral liver disease, e.g. chronic hepatitis C.

### DETAILED DESCRIPTION OF THE INVENTION

The person skilled in the pertinent art is fully enabled to select relevant test models to prove the beneficial effects mentioned herein of excess iron removal on liver disease. The pharmacological activity of such a compound may, for example, be demonstrated by means of the Examples described below, by in vitro tests and in vivo tests or in suitable clinical studies. Suitable clinical studies are, for example, open-label non-randomized, dose escalation studies of iron removal in patients with liver disease, as well as randomized, double-blind, placebo-controlled trials of iron removal in patients with liver disease.

The effective dosage of Compound I may vary depending on the pharmaceutical composition employed, on the mode of administration, the degree of iron excess present in the individual, the type of the liver disease being treated, or the severity of liver disease. The dosage regimen is selected in accordance with a variety of further factors including the renal and hepatic function of the individual. A physician, clinician or veterinarian of ordinary skill can readily determine and prescribe the effective amount of compound required to produce iron deficiency or near iron deficiency and thereby achieve therapeutic benefit.

Depending on age, individual condition, mode of administration, and the clinical picture in question, effective doses, for example daily doses of Compound I of 100 to 3000 mg of the active moiety are administered to warm-blooded animals, e.g. human, of about 70 kg body weight, e.g. 5 to 40 mg/kg of body weight/day. Preferably, the warm-blooded animal is a human. Compound I can be administered at the following dosage 5 to 40 mg/kg/day. In children the dosage is preferably 5 to 40 mg/kg of body weight/day. Daily doses of Compound I are for example 100 to 3000 mg of active moiety administered per day to a warm-blooded animal, e.g. a human. For patients with an inadequate response to daily doses, dose escalation can be safely considered and patients may be treated as long as they benefit from treatment and in the absence of limiting toxicities.

Ribavirin, marketed e.g. under the Trademarks, e.g. Copegus®; Rebetol®; Ribasphere®; Vilona®, Virazole®, can be administered according to the manufacturer's instructions, or e.g. at a dosage of about 200 mg up to about 1200 mg per day. Ribavirin is an oral medication.

Ribavirin can be given twice a day in 200-mg capsules for a total daily dose based upon body weight. The standard dose of ribavirin can be, e.g. 1,000 mg, for patients who weigh less than 75 kilograms (165 pounds) and, e.g. 1,200 mg for those who weigh more than 75 kilograms. In certain situations, an 800-mg dose (400 mg twice daily) can be recommended.

Interferon are for example, Interferon alfa-2a (Roferon-A; Hoffmann-La Roche), inteferon alpha-2b (Intron-A; Schering-Plough) and interferon alfacon-1 (Infergen; Intermune), and peginterferon alpha, sometimes called pegylated interferon, such as for example peginterferon alpha-2b (Peg-Intron; Schering-Plough) and peginterferon alpha-2a (Pegasys; Hoffmann-La Roche), Omega interferon (Intarcia), Multiferon (Viragen), Medusa Interferon (Flamel Technologies) and Albuferon (Human genome Sciences). Peginterferon alfa-2a can be given, e.g. subcutaneously, e.g. in a fixed dose, e.g. of 180 micrograms (mcg) per week. Peginterferon alfa-2b can be administered, e.g. subcutaneously weekly in a weight-based dose, e.g. of 1.5 mcg per kilogram per week, e.g. in the range of 75 to 150 mcg per week.

Interferon can be administered at a dosage of from 1 to 10 million units per day, e.g. depending on the body weight. Interferon can be administered e.g. once per day for 2 weeks followed by 3 times per week, or e.g. 3 times per week. Peginterferon alpha can be administered, e.g. once a week.

The invention relates to a method for administering to a human subject suffering from liver disease related to causes such as chronic hepatitis C infection or non-alcoholic steatohepatitis a pharmaceutically effective amount of Compound I once daily.

The invention relates to a method for administering to a human subject suffering from liver disease related to causes such as chronic hepatitis C infection or non-alcoholic steatohepatitis a pharmaceutically effective amount of Compound I and a biologic response modifier, e.g. an interferon, e.g. selected from the group comprising Interferon alfa-2a, interferon alpha-2b, interferon alfacon-1, peginterferon alpha-2b or peginterferon alpha-2a and/or a nucleoside anti-metabolite, e.g. selected from the group comprising ribavirin, viramidine or valopicitabine.

The invention relates especially to such method wherein a daily dose of 50 to 4000 mg of Compound I is administered to an adult or child. In the case of hepatitis C infection the administration of Compound I may be concomitant with or be followed by administration of anti-viral agents such as a biologic response modifier, e.g. an interferon, e.g. alpha-interferon and/or a nucleoside anti-metabolite, e.g. viramidine, valopicitabine or ribavirin.

The invention may be particularly relevant for the removal of iron from individuals who have liver disease benefiting from removal of iron who cannot be treated with phlebotomy because of accompanying anemia or other contraindications. In addition, the invention may be highly relevant for patients with hepatitis C unresponsive to standard anti-viral therapies.

The invention also relates to a method for administering to a human subject suffering from liver disease, a pharmaceutically effective amount of Compound I once daily on an intermittent basis, preferably fourteen days or two weeks out of every second or third month or seven days out of every month. The invention relates especially to such method wherein a daily dose of 50 to 4000 mg, preferably 1000 mg, of Compound I is administered to an adult or child.

The invention pertains to a:

use of Compound I of the following formula

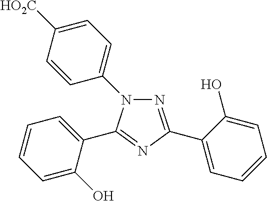 [SMILES: O=C(O)c1ccc(-n2nc(-c3ccccc3O)nc2-c2ccccc2O)cc1]

for the manufacture of a medicament for the treatment of liver disease in which iron plays a role in pathogenesis,

use of Compound I for the manufacture of a medicament for the treatment of liver disease in which iron plays a role in pathogenesis wherein the liver disease is chronic hepatitis C or non-alcoholic steatohepatitis,

use of Compound I for the manufacture of a medicament for the treatment of liver disease in which iron plays a role in pathogenesis, e.g. chronic hepatitis C or non-alcoholic steatohepatitis, wherein the iron state achieved by the treatment is a state of deficiency or near-deficiency,

a use of Compound I according for the preparation of a medicament for the treatment of a liver disease in which excess iron plays a role in pathogenesis.

use as mentioned above wherein Compound I is administered at a daily dose corresponding to 50 mg to 4000 mg of Compound I.

method of treating a mammal suffering from liver disease in which iron plays a role in pathogenesis that comprises administering to said mammal in need of such a treatment a dose, effective in removing excess iron, of Compound I.

combination comprising Compound I and a biologic response modifier, e.g. an interferon, e.g. selected from the group comprising Interferon alfa-2a, interferon alpha-2b, interferon alfacon-1, peginterferon alpha-2b or peginterferon alpha-2a, and/or a nucleoside anti-metabolite.

combination comprising Compound I and a biologic response modifier, e.g. an interferon, e.g. selected from the group comprising Interferon alfa-2a, interferon alpha-2b, interferon alfacon-1, peginterferon alpha-2b or peginterferon alpha-2a, and/or a nucleoside anti-metabolite, e.g. selected from the group comprising ribavirin, viramidine or valopicitabine.

combination comprising Compound I and a biologic response modifier, e.g. an interferon, e.g. selected from the group consisting of Interferon alfa-2a, interferon alpha-2b, interferon alfacon-1, peginterferon alpha-2b and peginterferon alpha-2a, and/or a nucleoside anti-metabolite, e.g. selected from the group consisting of ribavirin, viramidine and valopicitabine.

combination comprising Compound I and a biologic response modifier, e.g. an interferon, e.g. selected from the group comprising Interferon alfa-2a, interferon alpha-2b, interferon alfacon-1, peginterferon alpha-2b or peginterferon alpha-2a, and/or a nucleoside anti-metabolite, e.g. selected from the group comprising ribavirin.

a use of a combination comprising Compound I and a biologic response modifier, e.g. an interferon, e.g. selected from the group comprising Interferon alfa-2a, interferon alpha-2b, interferon alfacon-1, peginterferon alpha-2b or peginterferon alpha-2a, and/or a nucleoside anti-metabolite, e.g. selected from the group comprising ribavirin, viramidine or valopicitabine for the preparation of a medicament for the treatment of chronic hepatitis C patient non responsive to standard therapy.

a use of a combination comprising Compound I and a biologic response modifier, e.g. an interferon, e.g. selected from the group consisting of Interferon alfa-2a, interferon alpha-2b, interferon alfacon-1, peginterferon alpha-2b and peginterferon alpha-2a, and/or a nucleoside anti-metabolite, e.g. selected from the group consisting of ribavirin, viramidine and valopicitabine for the preparation of a medicament for the treatment of chronic hepatitis C patient non responsive to standard therapy.

The following examples are given to illustrate the present invention. It should be understood, however, that the invention is not to be limited to the specific conditions or details described in these examples.

### EXAMPLES

### Example 1

### Clinical Study Demonstrating the Efficiency of Iron Chelation of Compound I

A randomized, double-blind, placebo-controlled, dose-escalation trial of Compound I in 24 adult β-thalassemia patients in which the safety, tolerability, PK and cumulative iron balance of 12 days of Compound I are assessed (10 mg/kg (n=5), 20 mg/kg (n=6), 40 mg/kg (n=7), and placebo (n=6).

Compound I is rapidly absorbed and persisted in the blood over the entire interval when it is administered. Exposure (Cₘₐₓ and AUC) to Compound I increases slightly over-proportionally with the Compound I dose after single dose administration, but is seen to be approximately proportional during steady state. At pharmacokinetic steady state Cₘₐₓ is approximately 25% to 40% higher and the exposure to Compound I is 1.8 to 2.2 times higher than after a single dose at all dose levels. The mean elimination half-life t_{1/2} of both Compound I and its iron complex tend to be longer at steady state than after a single dose. Overall, at steady state the t_{1/2} of Compound I is approximately 12 to 13 hours and the t_{1/2} of the iron complex is generally longer (from 12 to 21 hours). Urinary excretion of Compound I and the iron complex is very low at all collection intervals (between 0.04% and 0.15% of the Compound I dose).

Iron balance studies in this trial demonstrated a dose-dependent increase in iron excretion, almost entirely in the feces. Efficiency of chelation is based on average daily net iron excretion, and is calculated as the ratio between the amount of iron that could theoretically be chelated, and the amount of iron actually excreted, relative to body weight. The theoretical amount of iron is obtained from the consideration that two molecules of Compound I are needed to chelate a single atom of iron. The molecular weight of Compound I being 373.4, and that of iron 55.85. The efficiency is therefore calculated as:

Efficiency=(Fe_{excr}*2*373.4)/(Dose_{Compound}×55.85)×100%)

Dose_{Compound} and Fe_{excr} are given in mg/kg body weight.

Negative iron balance is achieved at all 3 doses of active drug, and averaged approximately 0.127 mg/kg/day at the 10 mg/kg dose, 0.343 mg/kg/day at the 20 mg/kg dose, and 0.564 mg/kg/day at the 40 mg/kg dose. Significant variability is seen in the 40 mg/kg dose cohort. The observed efficiencies of chelation are 16% (10 mg/kg dose group), 22% (20 mg/kg), and 15% (40 mg/kg), see e.g. Nisbet-Brown et al., Lancet. 361:1597-1602.

### Example 2

### Clinical Study Demonstrating the Safety and Efficacy of Iron Reduction Therapy with Compound I

This clinical study is a two part trial examining the ability of daily doses of Compound I administered at 5 to 40 mg/kg to reduce serum ferritin levels, a marker of body iron stores, to less than 100 mcg/L. In the first part of the trial an optimal safe and effective dose is selected, and in the second part of the trial this dose of Compound I in mg/kg given daily and an approximately similar dose given in mg daily is compared to the safety and efficacy of phlebotomy for iron reduction therapy.

### Example 3

### Compound I Relieves Spontaneous Hepatitis in LEC Rats

Long-Evans cinnamon (LEC) rat is a mutant strain displaying hereditary hepatitis and spontaneous liver cancer. Compound I has been tested for efficacy on acute hepatitis in LEC rat model.

Methods: Compound I was administered orally to male LEC rats by gavage on does of 0, 14 and 28 mg/kg/day, starting at 6-week-old and continuing till 18-week-old. Each four rats were sacrificed on 9, 12, 14, 16 and 18-week-old in Compound I—treated groups and control group.

On sacrificing, peripheral blood was collected for monitoring the biochemical markers, including the serum alanine transaminase (ALT). Liver tissue were histologically examined.

Results: In non-treated group rats (control group), serum ALT started to increase from 16-week-old and reached 250 UI/L at 18-week-old. Mean±SD (standard deviation) serum ALT level at 18-week-old in the Compound I-treated groups was significantly lower than those in the control group. Hepatic iron accumulation assessed by Prussian blue staining was markedly reduced in the Compound I treated groups as compared to the control group. Compound I is effective to relive iron-induced acute hepatitis in LEC rats.

### Example 4

### Testing on an Rat Model of Hepatitis

Long-Evans cinnamon (LEC) rat is a mutant strain displaying hereditary hepatitis and spontaneous liver cancer. It is tested whether Compound I has a favorable effect on the development of hepatitis in LEC rat model.

Methods and Material:

1) Species & Number of Animals:

Long-Evans Cinnamon (LEC) rats (n=45/group). Each six animals will be sacrificed on 12, 13, 14, 16, 20, 24th week.

2) Method of Administration: Oral

3) Dosage and Duration of Administration: 14 and 28 mg/kg for maximum 24 weeks

### Example 5

### Compound I Improves the ALT Human Liver Values

ALT—(alanine aminotransferase also called SGPT, i.e. Serum Glutamic-Pyruvic Transaminase)—is a specific marker for liver damage. The ALT is an enzyme that is produced in the liver cells, i.e. hepatocytes; ALT is more specific for liver diseases than some of the other enzymes. It is generally increased in situations where there is damage to the liver, e.g. hepatitis, e.g. damage of the cell membranes. In normal patients with no liver damage, the ALT value is around zero.

Iron overloaded patients are developing liver injuries that lead to elevated ALT values. The enclosed results show that Compound I is useful to bring the ALT level back to a baseline level value in patients having elevated ALT levels.

Patients were iron-overloaded patients and were treated with different doses of Compound I for one year.

ALT was measured according to standard biomedical techniques, e.g. using the International Federation of Clinical Chemistry reference method as described in Brinkmann T, Dreier J, Diekmann J, Gotting C, Klauke R, Schumann G, Kleesiek K. Alanine aminotransferase cut-off values for blood donor screening using the new International Federation of Clinical Chemistry reference method at 37 degrees C. Vox Sang. 2003 85(3):159-64.

The enclosed results show that an appropriate dosing of Compound I results in patients having ALT parameters kept at the baseline value of ALT. i.e. at an ALT value not than the baseline ALT value.

The baseline ALT value is defined as the patient ALT value determined for the patient at the stage of enrollment in the clinical trial, i.e. the ALT baseline value is the ALT value of the patient before starting Compound I treatment.

The patients received doses of Compound I, their iron body content decreased, at an appropriate dosing of Compound I (see below, ALT values for the following doses 20 and 30 mg/kg of body weight/day). The ALT values are kept down at around the baseline value or improved to below the baseline value.

TABLE 1
ALT values of thalassemia patients after one year of
treatment with Compound I at different dosages (separate
trial as compared to the results in Table 1).

| Compound I | Number of patients | Mean of ALT (Units/liter) |
|---|---|---|
| 5 mg/kg/day | 2 | 35.25 |
| 10 mg/kg/day | 8 | 26.62 |
| 20 mg/kg/day | 21 | 3.17 |
| 30 mg/kg/day | 52 | −23.56 |

TABLE 2
ALT values of rare anemia patients after one year
of treatment with Compound I at different dosages

| Compound I | Number of patients | Mean of ALT (Units/liter) |
|---|---|---|
| 5 mg/kg/day | 4 | 67.42 |
| 10 mg/kg/day | 10 | 14.22 |
| 20 mg/kg/day | 24 | −3.27 |
| 30 mg/kg/day | 41 | −14.19 |

### Example 6

Compound I is administered to patients with chronic viral hepatitis C, e.g. genotype 1, who are non-responders or non sustained-responders to therapy including interferon, e.g. pegylated interferon and ribavirin.

2 to 3 different doses of Compound I are tested.

Number of patients per group: 8-12

### Example 7

Patients are administered:

combination of Compound I with a biologic response modifier, e.g. interferon alpha,

combination of compound I with a biologic response modifier, e.g. interferon alpha and a nucleoside anti-metabolite, e.g. ribavirin,

combination of Compound I with ribavirin.

What is claimed is:

1. A method of treating a patient suffering from liver disease, said patient not treatable with phlebotomy or a unresponsive to anti-viral therapy, comprising administering an effective amount of Compound I of the following formula:

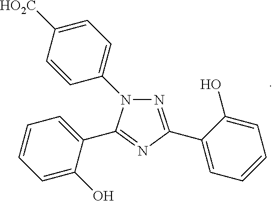 [SMILES: O=C(O)c1ccc(-n2nc(-c3ccccc3O)nc2-c2ccccc2O)cc1]

2. The method according to claim 1 wherein the liver disease is chronic hepatitis C, non-alcoholic steatohepatitis or non-alcoholic fatty liver disease.

3. The method according to claim 2 wherein the liver disease is chronic hepatitis C

4. The method according to claim 1 wherein the iron is reduced to a state of deficiency.

5. The method according to claim 1 wherein Compound I is in the form of a dispersible tablet.

6. The method according to claim 1 wherein Compound I is in crystalline form A.

7. The method according to claim 1 wherein Compound I is administered at a daily dose corresponding to 50 mg to 4000 mg of Compound I.

8. The method according to claim 1 wherein Compound I is administered once daily for at least two weeks every 2 or 3 months.

9. The method according to claim 1 wherein Compound I is administered once daily for at least 7 days per months.

10. The method according to claim 1 wherein Compound I is effective in removing excess iron.

11. A method of treating a patient suffering from liver disease in which iron plays a role in pathogenesis-comprising administering an effective amount of Compound I in crystalline form A of the following formula:

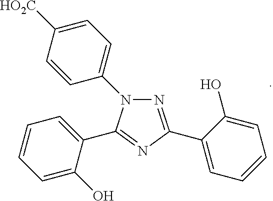 [SMILES: O=C(O)c1ccc(-n2nc(-c3ccccc3O)nc2-c2ccccc2O)cc1]

12. The method according to claim 1 wherein the liver disease is chronic hepatitis C, non-alcoholic steatohepatitis or non-alcoholic fatty liver disease.

13. The method according to claim 1 wherein Compound I is in the form of a dispersible tablet and wherein the iron is reduced to a state of.

14. The method according to claim 1 wherein Compound I is administered at a daily dose corresponding to 50 mg to 4000 mg of Compound I.
